(19) United States
(12) Patent Application Publication
(10) Pub. No.: US 2004/0171072 A1
(43) Pub. Date: Sep. 2, 2004

(54) Solid support based on selenium useful in solid phase synthesis

(75) Inventors: Thomas Ruhland, Valby (DK); Kim Andersen, Virum (DK); Henrik Pedersen, Bronshoj (DK)

Correspondence Address: DARBY & DARBY P.C., 805 Third Avenue, New York, NY, 10022, US

(73) Assignee: H. Lundbeck A/S, Copenhagen-Valby (DK)

(21) Appl. No.: 10/619,931

(22) Filed: Jul. 14, 2003

Related U.S. Application Data
(63) Continuation of application No. 09/677,851, filed on Sep. 29, 2000, now abandoned.
Application No. 09/677,851 is a continuation of application No. PCT/DK99/00179, filed on Mar. 26, 1999.

(30) Foreign Application Priority Data
Mar. 31, 1998 (DK) 0453/98

(51) Int. Cl. G01N 33/53; G01N 33/543; C08F 212/08

(52) U.S. Cl. 435/7.1; 436/518; 525/54.1; 525/333.3

(57) ABSTRACT

This invention relates to novel solid support based on selenium and a method for the preparation thereof. The solid support is useful in solid phase synthesis of organic compounds including combinatorial libraries of compounds.

### FIELD OF THE INVENTION

The present invention relates to a novel solid support, a method for the preparation thereof and the use of the solid support in solid phase synthesis of organic compounds including combinatorial libraries of compounds.

### BACKGROUND OF THE INVENTION

The use of solid phase synthesis for the preparation of combinatorial libraries has been going on for some years and a number of technologies for solid phase synthesis have been described in the recent years, e.g Thompson, L. A.; Ellman, J. A. Chem. Rev. 1996, 96, 555-600, Früchtel, J. S.; Jung, G. Angew. Chem. 1996, 108, 1946, Hermkens, P. H. H.;. Ottenheijm, H. C. J.; Rees, D. Tetrahedron 1996, 52, 4527-4554, Balkenhohl, F.; von dem Bussche-Hünnefeld, C.; Lansky, A.; Zechel, C. Angew. Chem. Int. Ed. Engl. 1996, 35, 2288-2337, Nefzi, A.; Ostresh, J. M.; Houghten, R. A. Chem. Rev. 1997, 97, 449-472 and Hermkens, P. H. H.; Ottenheijm, H. C. J.; Rees, D. C. Tetrahedron 1997, 53, 5643-5678.

During this period a variety of polymer supports and linkers was introduced together with a wide range of methods of attachment, possible types of reactions and methods of cleavage using these polymer supports and linkers.

When a solid phase synthesis strategy for a combinatorial library is considered, the key question is the most suitable choice of solid support. The solid support has to be stable to all reaction conditions during the synthesis and, after assembly is complete, it must liberate the final molecules selectively and without causing degradation or side products.

Among the versatile solid supports so far developed, the solid supports with so called traceless linkers are particularly attractive because molecules are directly attached and the liberated final compounds bear only those functional groups which have been chosen for e.g. biological activity.

In the field of C—H bond forming traceless linkers some linkers have been described. For aromatic C—H bond formation, polystyrene based silicon was described by Plunkett, M. J.; Ellman, J. A. J. Org. Chem. 1997, 62, 2885-2893, Plunkett, M. J.; Ellman, J. A. J. Org. Chem. 1995, 60, 6006-6007, Chenera, B.; Finkelstein, J. A.; Veber, D. F. J. Am. Chem. Soc. 1995, 117, 11999-12000, Han, Y.; Walker, S. D.; Young, R. N. Tetrahedron Lett. 1996, 37, 2703-2706, Boehm, T.; Showalter, H. D. H J. Org. Chem. 1996, 61, 6498-6499, Woolard, F. X.; Paetsch, J.; Ellman, J. A. J. Org. Chem. 1997, 62, 6102-6103 and germanium-linking strategies have been developed by Plunkett, M. J.; Ellman, J. A. J. Org. Chem. 1997, 62, 2885-2893. For aliphatic C—H bond formation, polyethylene glycol based sulphur-linking strategies have been reported by Sucholeiki, I. Tetrahedron Lett. 1994, 35, 7307-7310, Jung, K. W.; Zhao, X.-Y.; Janda, K. D. Tetrahedron Lett. 1996, 37, 6491-6494 and Jung, K. W.; Zhao, X.-Y.; Janda, K. D. Tetrahedron 1997, 53, 6645-6652.

In the latter strategies, attachments were indirectly achieved in a multistep procedure and by the use of an auxiliary amide-containing spacer, which is sensitive in a variety of reaction conditions, e.g. reducing reagents like LiAlH₄. The final compounds were liberated upon C—S bond cleavage by hydrogenolysis with H₂/Raney-nickel, Jung, K. W.; Zhao, X.-Y.; Janda, K. D. Tetrahedron Lett. 1996, 37, 6491-6494 and Jung, K. W.; Zhao, X.-Y.; Janda, K. D. Tetrahedron 1997, 53, 6645-6652 or by homolysis either with tributylstanane/AIBN and at elevated temperature, Jung, K. W.; Zhao, X.-Y.; Janda, K. D. Tetrahedron Lett. 1996, 37, 6491-6494 or under irradiation, Sucholeiki, I. Tetrahedron Lett. 1994, 35, 7307-7310. Hydrogenolysis with H₂/Raney-nickel is reported to proceed smoothly but its application is neither very. suitable for automated solid phase synthesis nor compatible to, reduction-sensitive functional groups e.g. alkynes or epoxides. Homolysis turned out either to be very slow with tributylstanane/AIBN or questionably selective under irradiation.

In Scaiano, J. C.; Schmid, P.; Ingold, K. U. J. Organomet. Chem. 1976, 121, C4, Liotta, D., Organoselenium Chemistry; John Wiley & Sons, Inc.: New York, 1987 and Davies, A. G. Organotin Chemistry; V C H Verlagsgesellschaft: Weinheim, 1997, it is mentioned that homolysis with tributylstanane/AIBN of aryl alkyl selenides proceeds faster than for the corresponding sulfides, suggesting alkanes to be more smoothly released from resin bound selenides than from resin bound sulfides previously described.

Derivatives of polystyrene bound selenium have been described by W. Heitz for the use as resin-bound oxidation reagents, Kato, M. German Patent Application No. DE 2649163, 1976, Kato, M.; Michels, R.; Heitz, W. Polymer Letters Edition 1976, 14, 413-415 and Michels, R.; Kato, M.; Heitz, W. Makromol. Chem. 1976, 177, 2311-2320. It was known that reactions using selenium in solution were accomplished with a certain degree of toxicity. Therefore, the purpose of using this preparation of polystyrene bound selenium as a reagent was to exclude the known toxicity and thereby be able to use selenium reagents in the reactions.

Consequently, there is a need for a solid support where attachment can be achieved in a simple way, where the linker will be stable to a broad variety of reaction conditions during the synthesis and where the linker, after assembly is complete, will be able to liberate the final molecules selectively.

### SUMMARY OF THE INVENTION

It has now been discovered that the instant novel solid support based on selenium is useful in the solid phase synthesis of organic compounds including libraries of compounds for biological or physical testing.

One aspect of this invention relates to a solid support useful in solid phase synthesis of a combinatorial library of organic compounds wherein the solid support is consisting of polystyrene bound selenium.

Another aspect of this invention relates to a novel solid support composition having the formula PS—Se—B(OR)₃⁻M⁺, wherein PS is polystyrene; R is C₁-C₆ alkyl; M is Li, Na, K, Zn or Cs.

Another aspect of this invention relates to the process of preparing the solid support described above comprising the steps of

a) lithium-bromine exchange of bromopolystyrene with BuLi

b) suspension in a non-protic solvent and treatment with selenium, wherein the polar non-protic solvent is dimethoxyethan, diethylether, THF, toluene or dioxane, preferred THF

c) treatment with Mⁿ⁺(BH₄)⁻ₙ in ROH

And a solid support prepared by the process.

Yet another aspect of the invention relates to a method for synthesizing organic compounds including single compounds and combinatorial libraries of compounds on a solid support wherein the solid support is polystyrene bound selenium and the method comprises the steps of:

a) attachment by direct loading to the solid support of a compound of formula R¹R²R³CX, wherein X is a halogenide or a substituted alkyl or aryl sulfonate; R¹, R² and R³ are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl, provided that at least one of R¹, R² and R³ is not hydrogen

b) additional modification of the R¹, R² or R³-groups by a synthesis sequence comprising one or more reactions being compatible with aryl alkyl selenides.

c) cleavage with formation of aliphatic C—H bond on final compound of formula, R¹′R²′R³′CH wherein R¹′, R²′ and R³′ are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl

d) optionally purification by solid phase extraction

Yet another aspect of the invention relates to another method for synthesizing organic compounds including single compounds and combinatorial libraries of compounds on a solid support wherein the solid support is polystyrene bound selenium, and the method comprises the steps of:

a) attachment by direct loading to the solid support of a compound of formula XCR¹R²—CHR³R⁴, wherein X is a halogenide or a substituted alkyl or aryl sulfonate; R¹, R² , R³ and R⁴ are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl, provided that at least one of R¹, R², R³ and R⁴ is not hydrogen

b) additional modification of the R¹, R², R³ and R⁴-groups by a synthesis sequence comprising one or more reactions being compatible with aryl alkyl selenides.

c) cleavage under oxidative conditions under β-elimination process on final compounds of the general structure CR^{1′}R^{2′}═CR^{3′}R^{4′}, wherein R^{1′}, R^{2′}, R^{3′}and R^{4′}are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl

d) optionally purification by solid phase extraction

The compounds can be attached by the methods of the invention in a single step to the solid support by direct loading without the requirement of an auxiliary spacer and are subsequently cleaved selectively under mild conditions.

Based upon the disclosure herein, it will be clear to one of ordinary skill in the art that the solid support of the invention may be useful in many possible synthetic approaches creating the combinatorial libraries. The overall approach can be applied to solid-phase synthesis of many classes of organic compounds under a broad variety of reaction conditions compatible with aryl alkyl selenides.

### DETAILED DESCRIPTION OF THE INVENTION

The term “solid phase synthesis” is used herein to mean one or a series of chemical reactions used to prepare organic compounds including combinatorial libraries of organic compounds, wherein the chemical reactions are performed on a compound to be derivatized, which compound is bound to a polymer support through a linkage until the compound is cleaved to the final compound.

The term “combinatorial library” is used herein to mean a collection of single compounds or mixtures of compounds prepared by a common synthesis sequence, the structural variation of the compounds are obtained by variation of the diversifying reagent or reagents in each reaction step of the synthesis sequence.

The novel composition of the polymer support and the linker of this invention are described herein with the term “solid support”. The solid support can be illustrated by the following figure

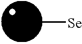

Furthermore, the term “polystyrene bound selenium” is used herein to mean the polymer supports according to this invention, wherein selenium is bound to the polystyrene. Additionally, the compounds to be derivatized are bound to the selenium.

The term “polystyrene” refers to polymerised styrene including polymerised styrene crosslinked by the addition of divinylbenzene.

The term “final compounds” is used herein to mean the compounds that before the cleavage from the solid support, were bound to the selenium.

The preparation of the final compounds using solid phase synthesis is consisting of the attachment of compounds to the solid support, followed by additional modification of the compounds by a synthesis sequence comprising one or more reactions and finally cleavage of the final compounds from the solid support.

The attachment according to this invention is by direct loading. The term “direct loading” is used herein to mean that the compounds are attached in a single step to the solid support without the requirement of an auxiliary spacer.

Preferred attachment according to this invention is by means of polystyrene bound selenium by alkyl halogenide or an alkyl or aryl sulfonate.

The additional modification of the compounds by a synthesis sequence comprising one or more reactions being compatible with aryl alkyl selenides. The compatibility of aryl alkyl selenides is well known to the chemist skilled in the art.

The broad acceptance of selenides towards various reaction conditions makes the solid supports of the invention very suitable in solid phase synthesis of organic compounds.

The cleavage is performed under the formation of an aliphatic C—H bond on the final compound. The term “formation of aliphatic C—H bond” is used herein to mean that the bond between a selenium atom and an aliphatic carbon atom is replaced by a bond between a hydrogen atom and an aliphatic carbon atom.

Preferred methods of cleavage according to this invention are radical hemolysis with trialkyl stannanes and a radical initiator such as AIBN.

Oxidation with subsequent β-elimination is used herein to mean oxidation with an oxidative agent such as sodium periodide, H₂O₂ or m-CPBA, followed by spontaneous cleavage under β-elimination under the formation of a double bond in the final compound.

The term “solid phase extraction” is used herein to mean purification by chromatography using ion exchange resins, silicagel or derivatized silicagel or aluminium oxide support preferably by parallel or automated methods

The term C₁-C₆ alkyl refers to such branched or unbranched groups having from one to six carbon atoms inclusive. Exemplary of such groups are methyl, ethyl, 1-propyl, 2-propyl, 1-butyl, 2-butyl, 2-methyl-2-propyl, 2-methyl-1-propyl, or the like, preferably ethyl.

M is preferably Na.

As used herein the term alkyl refers to a C₁-C₂₀ straight chain or branched alkyl group and similarly alkenyl and alkynyl mean a C₂-C₂₀ straight chain or branched hydrocarbon group having one or more double bonds or triple bonds, respectively. The term cycloalkyl designates a carbocyclic ring having 3-8 carbon atoms, inclusive, or a bicyclic or tricyclic carbocycle, such as adamantyl.

The terms aryl and heteroaryl refer to a mono- or bicyclic carbocyclic or heterocyclic aromatic group, such as phenyl, indolyl, thienyl, furanyl, pyridyl, thiazolyl, benzofuranyl, benzothienyl, benzisothiazolyl and benzisoxazolyl.

The term heteroatom is used herein to mean an oxygen atom, a sulfur atom or a nitrogen atom. Accordingly, the terms heteroalkyl and heteroaryl are used herein to mean alkyl and aryl comprising one or more heteroatoms.

Halogenide means chloro, bromo or iodo, whereas halogen means fluoro, chloro, bromo or iodo.

The term “optionally substituted” is used herein to mean that the moieties may or may not be substituted with one or more of various functional groups including, alkyl, alkenyl, alkynyl, aryl, cycloalkyl, heteroaryl, arylalkyl, heteroarylalkyl, halogen, NO₂, —OR⁵, —N(R⁵)₂, —NHC(O)R⁵, SO₂N(R⁵)₂, —CO₂R⁵ or —CON(R⁵)₂, wherein R⁵ is hydrogen, C₁-C₆-alkyl, aryl, arylalkyl, heteroalkyl, heteroarylalkyl, heterocyclic or heterocyclicalkyl.

The preparation according to the present invention of the resin bound selenium was achieved by the procedure described below.

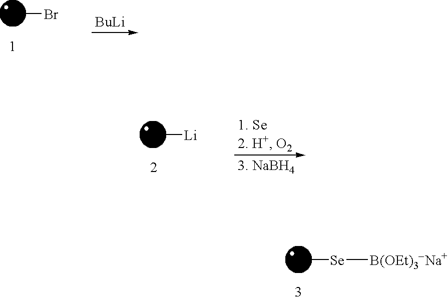

Bromopolystyrene 1 was obtained through thallium acetate catalysed bromination of commercially available polystyrene (crosslinked with 1% divinylbenzene), Farrall, M. J.; Fréchet, J. M. J. J. Org. Chem. 1976, 41, 3877-3882. The loading of the resulting resin was determined by elemental analysis for bromine to be 3.7 mmol/g. After lithium-bromine exchange with excess butyllithium (BuLi) in (1:1) hexane/toluene and subsequent removal of solvent by decantation, the lithiated polystyrene 2 was suspended in THF and treated with selenium-powder.

In order to liberate the resin from excess selenium, the mixture was treated several times with NaBH₄ in MeOH. After drying in vacuum an orange resin was obtained which displayed no visible swelling properties in any solvent. It is very likely that Se—Se bonds were formed by oxidation under air exposure during working up, Farrall, M. J.; Fréchet, J. M. J. J. Org. Chem. 1976, 41, 3877-3882. The Se—Se bond forming causes high degree of crosslinking which explains the poor swelling, Hodge, P. Chemical Society Reviews 1997, 26, 417-424. In EtOH however, within 1-2 h after addition of NaBH₄, the resin became distinctly swollen and almost colourless. Simultaneously an intensive-hydrogen-generation occurred.

Recently Miyashita, M.; Suzuki, T.; Hoshino, M.; Yoshikoshi, A. Tetrahedron 1997, 53, 12469-12486 proved, that the reaction between diphenyldiselenide and NaBH₄ in EtOH results in the formation of hydrogen and the sodiumphenylseleno(triethyl)borate complex, Na[PhSeB(OEt₃)], but does not lead to sodium phenylselenide as previously proposed.

According to the knowledge of this reference, the analogous structure 3 was achieved for the polystyrene bound selenide anion after reduction with NaBH₄ in EtOH. This also explains the observation of strong hydrogen generation during reduction and the striking swelling property of the resin in EtOH.

In order to determine the loading of polystyrene bound selenium, resin 3 was converted into resin 4 through alkylation with chloro-N,N-dimethylacetamide using the reaction conditions described below

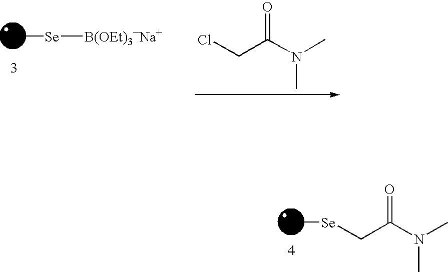

Elemental analysis for nitrogene revealed a loading of 1.8 mmol/g. No bromine was found by elemental analysis indicating that previously performed lithium-bromine exchange had gone to completion.

To illustrate the use of polystyrene bound selenium for solid phase synthesis a solid phase synthesis route of a [2×3]-sized alkylarylether library is outlined below in Scheme 3.

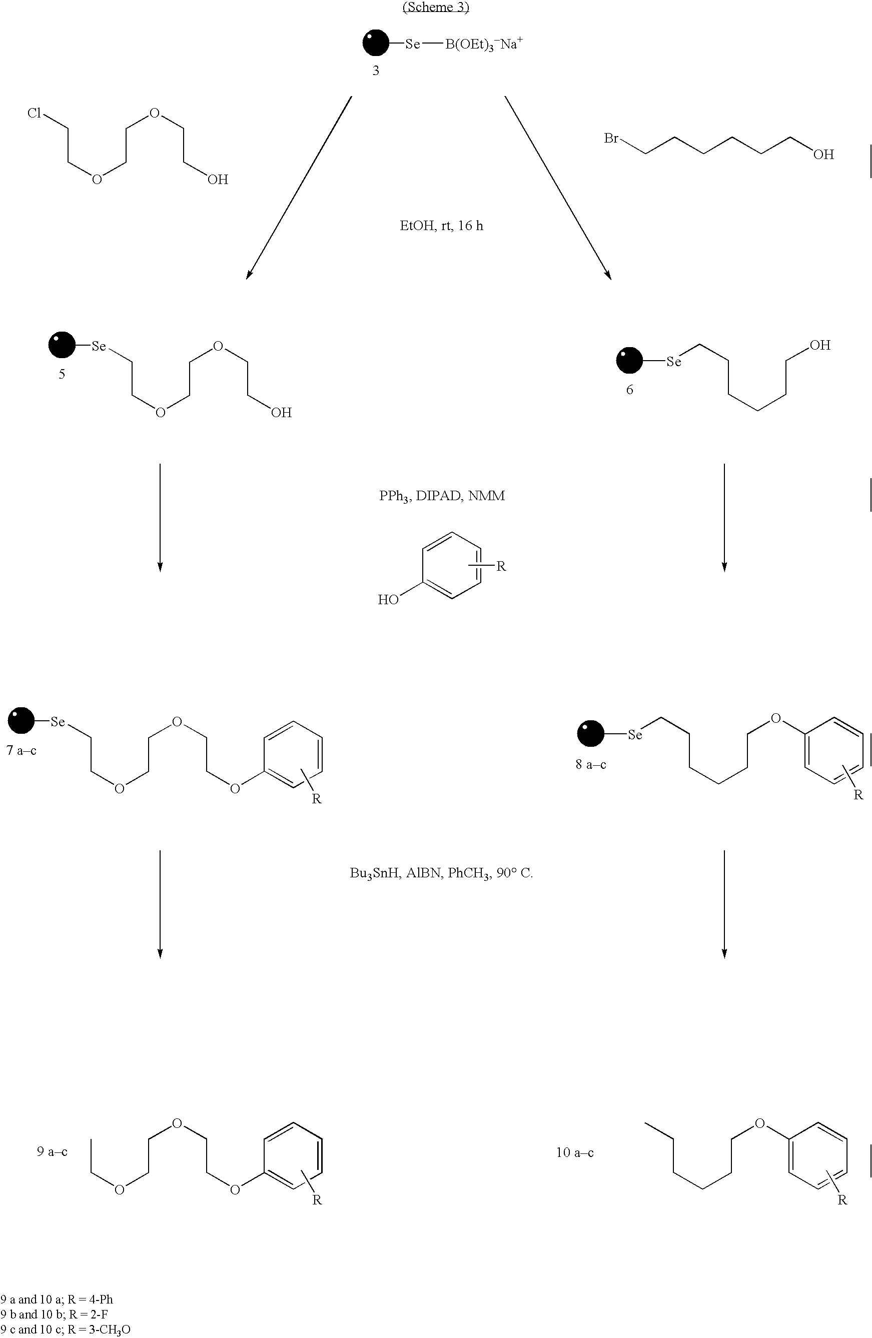

The attachment was illustrated and exemplified by alkylation of polystyrene bound selenium with 2-[2-[2-chloroethoxy)ethoxy]ethanol and 6-bromohexanol, respectively yielding resin bound alcohols 5 and 6. Prior to the alkylation, the resin was treated with NaBH₄ in EtOH to assure that crosslinking diselenides were reduced.

The reactions following the attachment were illustrated and exemplified by Mitsunobu ether bond formation. The reactions were carried out with triphenylphosphine, diisopropylazadicarboxylate (DIPAD) as reagents and with N-methyl-morpholine (NMM) as solvent, Richter, L. S.; Gadek, T. R. Tetrahedron Lett. 1994, 35, 4705-4706. Coupling of each of the resin bound alkohols 5 and 6 with 4-phenyl-, 2-fluoro-, and 3-methoxy-phenol furnished the six alkyl aryl ether 7a-c and 8a-c as single discrete compounds. No resin bound alcohols were detectable by HR-MAS ¹H-NMR indicating that the coupling went to completion.

Cleavage of the products was achieved with tributylstanane and a catalytic amount of AIBN in toluene at 90° C. for 12 h. HR-MAS ¹H-NMR analyses of the cleaved resin showed only very pure resin bound tributyltin selenide 11 and no traces of the intermediate resin bound aryl alkyl ethers revealing that the cleavage was quantitative.

The alkylarylethers were obtained in 57-83% yield and 78-88% purity (GC) after separation from the cleavage reagents by solid phase extraction. The automation of solid phase extraction makes it possible to purify large libraries prepared by this method.

### EXAMPLES

The general conditions for the experimental work were as described below.

Unless otherwise noted, starting materials were obtained from commercial suppliers and used without further purification. Tetrahydrofuran (THF) was destined under N₂ from sodium/benzophenone immediately prior to use. Flash column chromatography was carried out according to the procedure described by Still. For Flash column chromatography and for solid phase extraction, Scharlau 60 230-400 mesh silicagel (sorbil) was used. Thin layer chromatography (TLC) was performed on Merck 60 F₂₅₄ 0.25 μm silica gel plates. Unless otherwise stated, TLC—R_{ƒ} values given were determined with the solvent used for column chromatography. ¹H—NMR and ¹H- decoupled ¹³C-NMR spectra were recorded at 500.13 MHz and 125.67 MHz, respectively, on a Bruker Avance DRX 500 instrument. NMR spectra of polymer bound substances were recorded with a 4 mm ¹H/¹³C double resonance high resolution MAS probehead optimized for proton resonance and equipped with one axis pulsed field gradient coil. Unless otherwise noted, compounds were measured in deuterated chloroform (99.8%). Chemical shifts for ¹H NMR are reported in ppm with TMS as internal reference. Chemical shifts for ¹³C NMR and high resolution MAS NMR are reported in ppm relative to chemical shift of deuterated solvents. Coupling constants (J values) are in Hertz. Gas chromatography (GC) was performed on a Varian Star 3400 CX instrument using an injector temperature of 200° C., detector temperature of 325° C., gas flow of 4.9 mL/min at 65° C., a splitflow of 150 mL/min and a Restek Rtx-5 column with a length of 15 m, inner diameter of 0.32 mm and a crossbonded phase of 0.50 mm. A temperature gradient of 15 degrees/minute from 65° C. to 275° C. was used. High resolution mass spectra (HRMS) were performed with the peak matching method using a Varian MAT 311A mass spectrometer. Elemental analysis were performed with a Perkin-Elmer 2.400 CHN elemental analyser. Polystyrene for the preparation of bromopolystyrene according to the procedure described by Fréchet was purchased from Rapp Polymere GmbH (Tübingen, Germany) (no. H 1000, 100-200 mesh, crosslinked with 1% divinylbenzene).

### Example 1

Preparation of Resin-Bound Selenium

Bromopolystyrene (24.0 g, 2.94 mmol/g) was preswollen in dry toluene (200 mL) for 15 min and BuLi (100 mL, 160 mmol, 1.6 M in hexane) was added. The mixture was stirred for 2 h at room temperature and the resin was allowed to settle. After the solvents were carefully removed by decantation, BuLi (200 mL, 320 mmol, 1.6 M in hexane) and dry toluene (200 mL) were added. The suspension was heated at 60° C. for 3 h. After cooling to room temperature the solvent was removed by decantation without further washing. After cooling to 0° C. dry THF (250 mL) was added. Immediately afterwards selenium powder (25.1 g, 318 mmol, 100 mesh) was carefully added in small portions (1-2 g) under intensive stirring. The addition of selenium was complete within 3-5 min. After heating the black suspension at 50° C. for 12 h, the mixture was cooled to room temperature and filtered. The black residue was washed with THF (1×250 mL), methanol (1×250 mL), (10:20) 2N aqueous HCl/THF (1×250 mL) and water (3×250 mL). For safety reasons the excess selenium was removed from the residue only bit by bit. Small portions of the residue (2-3 g) were suspended in methanol (250 mL) in a 3L Erlenmeyer-flask and treated carefully in small portions with excess of fine granulated sodium borohydride (5 g, 132 mmol). (CAUTION: generation of heat, hydrogen and toxic sodium selenides). After the gas evolution ended, the resin was filtered and washed with methanol (1×250 mL). The procedure was repeated for each resin-fraction until the excess selenium was remarkably taken away. The combined fractions were suspended in methanol (500 mL) and treated with sodium borohydride (5 g, 132 mmol) as described above for each single portion. The procedure was repeated about 8-10 times. After every fourth treatment with sodium borohydride the resin was additionally washed with (10:20) 2N aqueous NaOH/THF (1×250 mL), water (1×250 mL), (10:20) 2N aqueous HCl/THF (1×250 mL), water (1×250 mL), THF (1×250 mL) and methanol (1×250 mL) (washing with methylene chloride should be omitted because the solvent could couple to the resin). Finally the pale yellow resin and sodium borohydride (5 g, 132 mmol) were refluxed in (200:10) ethanol/methanol (500 mL) for 2 h. The resin was filtered and washed as described above with the exception that THF (2×250 mL) was used in the last washing step. After drying in vacuo a orange resin (24.3 g) was obtained. The loading of the resin was calculated to be 1.84 mmol/g determined by elemental analysis for nitrogene after alkylation with chloro-N,N-dimethylacetamide

### Example 2

Alkylation of Resin Bound Selenium

N,N-Dimethylformylmethylselanyl polystyrene 4

The procedure for a typical experiment follows. Polymer bound selenium (50 mg) was suspended in (400:10) ethanol/methanol (0.5 mL) and treated with sodium borohydride (40 mg, 1 mmol) at room temperature. After approximately 1 h, gas and heat generation occurred and the resin became swollen and almost colourless. The mixture was stirred for approximately 3 h until the gas evolution stopped. The resin was allowed to settle and the above solution was removed by a pipette. After washing with ethanol under N₂ atmosphere (1×20 mL) the resin was treated with a chloro-N,N-dimethylacetamide (207 mg, 1.7 mmol) in ethanol (0.5 mL) and the mixture was stirred for 12 h at room temperature. The almost colourless resin was filtered, washed with ethanol (2×25 mL), water (2×25 mL), THF (1×25 mL), ethanol (1×25 mL), water (1×25 mL), acetone (1×25 mL) and methylene chloride (3×25 mL) and dried in vacuo at room temperature. Anal. Calcd.: found C, 67.97; H, 6.46; N, 2.22; Br,<0.1. According to the elemental analysis for N, a loading of 1.59 mmol/g was calculated for resin 4 which corresponds to a loading of 1.84 mmol/g for the initial loading of polystyrene bond selenium, assuming that the alkylation went to completion.

The resins 5 and 6 were prepared according to above described procedure by alkylation with with 2-[2-[2-chloroethoxy)ethoxy]ethanol and 6-bromohexanol, respectively.

### Example 3

Alkylarylether Synthesis by Mitsunobu Reaction

2-[2-[2-(3-Methoxyphenoxy)ethoxy]ethoxy]ethylselanyl polystyrene 7c

The procedure for a typical experiment follows. Resin-bound alkylalcohol 5 (500 mg, 0.74 mmol) was preswollen in 4-methylmorpholin (5 mL) for 5 min. Neat 3-methoxyphenol (571 mg, 4.60 mmol) and triphenylphosphine (1.21 g, 4.61 mmol) were added at room temperature. After complete dissolution, neat diisopropyl azodicarboxylate (930 mg, 4.60 mmol) was added in small portions over a period of 15 min at room temperature. After stirring of the suspension for 12 h at room temperature, the resin was filtered and subsequently washed with THF (3×10 mL), DMSO (2×10 mL), THF (2×10 mL), water (2×10 mL), methanol (2×10 mL), methylene chloride (3×10 mL) and dried in vacco at room temperature for 12 h. Resin 7c was calculated to have a loading of 1.28 mmol/g, assuming the Mitsunobu reaction went to completion.

The resins 7a, 7b, 8a, 8b and 8c were prepared according to above described procedure.

### Example 4

Homolytic Cleavage

1-[2-[2-(2-Ethoxy)ethoxy]ethoxy]-3-methoxybenzene 9c

The procedure for a typical experiment follows. Resin 7c (1.00 mg, 1.28 mmol) was preswollen in toluene (10 mL) for 5 min. Neat tributylstannane (1.62 g, 5.6 mmol) and AIBN (20 mg, 0.12 mmol) were added and the mixture was heated in a sealed tube to 90° C. for 12 h. After cooling to room temperature the resin was filtered and washed with THF (2×2 mL), acetone (1×2 mL) and methylene chloride (2×2 mL). The filtrates were combined and the solvents were evaporated in vacuo. The residue was purified by solid phase extraction using silicagel (6.2 g). Unpolar tin impurities were removed by washing the column with pure heptane. Elution with (150:10) heptane/ethyl acetate gave 213 mg (70%) of the desired product 9c as a clear oil (78% purity by GC, Rt=9.3 min). An analytical sample was obtained by flash chromatography (50:10 heptane/ethyl acetate) and subsequent microdestillation (0.1 mmHg, 90-95° C.). TLC (20:10 heptane/ethyl acetate): R_{f}=0.41. ¹H NMR: δ1.21 (t, 3H, J=7.0), 3.52 (q, 2H, J=7.0), 3.60 (t, 2H, J=4.8), 3.70 (t, 2H, J=4.8), 3.76 (s, 3H), 3.84 (t, 2H, J=4.9), 4.12 (t, 2H, J=4.9), 6.50 (m, 3H), 7.15 (t, 1H, J=8.1). ¹³C NMR: δ15.5, 55.6, 67.0, 67.8, 70.1, 70.2, 71.3, 101.6, 106.9, 107.1, 130.2, 160.5, 161.2. HRMS: calcd. for C₁₃H₂₀O₄ 240.1362, found 240.135. Anal. Calcd. for C₁₃H₂₀O₄: C, 64.98; H, 8.39, found C, 64.90; H, 8.65.

The following alkylarylethers 9a, 9b, 10a, 10b and 10c were prepared according to above described procedure.

### Example 5

Hexyloxy-3-methoxy-benzene 10c

This compound was synthesized from resin 8c (924 mg, 1.23 mmol) and 3-methoxyphenol as described in Example 4. Solid phase extraction (150:10 heptane/ethyl acetate) gave 146 mg (57%) of the desired product as a clear oil (87% purity by GC, Rt=8.0 min). An analytical sample was obtained by flash chromatography (300:10 heptane/ethyl acetate) and subsequent microdestillation (0.1 mmHg, 50-55° C.). TLC: R_{f}=0.32. ¹H NMR: δ0.90 (t, 3H, J=6.6), 1.33 (m, 4H), 1.44 (m, 2H), 1.76 (p, 2H, J=7.1), 3.76 (s, 3H), 3.91 (t, 2H, J=6.6), 6.46 (m, 1H), 6.48 (m, 2H), 7.15 (t, 1H, J=8.1). ¹³C NMR: δ14.5, 23.1, 26.2, 29.7, 32.0, 55.6, 68.4, 101.4, 106.5, 107.1, 130.2, 160.9, 161.3. HRMS: calcd. for C₁₃H₂₀O₂ 208.1463, found 208.145. Anal. Calcd. for C₁₃H₂₀O₂: C, 74.96; H, 9.68, found C, 74.69; H, 9.93.

### Example 6

1-{2-[2-(2-Ethoxy)ethoxy]ethoxy}-2-fluorobenzene 9b

This compound was synthesized from resin 7b (950 mg, 1.24 mmol) and 2-fluorophenol as described in Example 4. Solid phase extraction (50:10 heptane/ethyl acetate) gave 200 mg (72%) of the desired product as a clear oil (88% purity by GC, Rt=7.4 min). An analytical sample was obtained by flash chromatography (40:10 heptane/ethyl acetate) and subsequent microdestillation (0.1 mmHg, 70-75° C.). TLC: R_{f}=0.15. ¹H NMR: δ1.20 (t, 3H, J=7.0), 3.52 (q, 2H, J=7.0), 3.60 (t, 2H, J=4.8), 3.72 (t, 2H, J=4.8), 3.87 (t, 2H, J=5.0), 4.19 (t, 2H, J=5.0), 6.89 (m, 1H), 7.02 (m, 3H). ¹³C—NMR: δ15.9, 67.4, 69.9, 70.5, 70.7, 71.8, 116.4, 117.0 (d, J=18.3), 122.2 (d, J=6.8), 125.0(d, J=3.5), 147.8 (d, J=10.6), 153.7 (d, J=245.6). HRMS: calcd. for C₁₂H₁₇FO₃ 228.1162, found 228.118. Anal. Calcd. for C₁₂H₁₇FO₃: C, 63.14; H, 7.51, found C, 63.36; H, 7.35.

### Example 7

Hexyloxy-2-fluorobenzene 10b

This compound was synthesized from resin 8b (985 mg, 1.33 mmol) and 2-fluorophenol as described in Example 4. Solid phase extraction (heptane) gave 174 mg (67%) of the desired product as a clear oil (80% purity by GC, Rt=5.9 min). An analytical sample was obtained by flash chromatography (heptane) and subsequent microdestillation (15 mmHg, 95-105° C.). TLC: R_{f}=0.31. ^{1H NMR: δ}0.90 (t, 3H, J=6.9), 1.34 (m, 4H), 1.47 (m, 2H), 1.81 (p, 2H, J=7.1), 4.02 (t, 2H, J=6.6), 6.86 (m, 1H), 6.95 (t, 1H, J=7.8), 7.05 (m, 2H). ¹³C NMR: δ14.6, 23.2, 26.3, 29.9, 32.2, 70.1, 115.7, 116.8 (d, J=18.4), 121.5 (d, J=6.8), 124.8 (d, J=3.4), 147.9 (d, J=10.6), 153.6 (d, J=245.4). HRMS: calcd. for C₁₂H₁₇FO 196.1263, found 196.126.

### Example 8

1-{2-[2-(2-Ethoxy)ethoxy]ethoxy}-4-phenylbenzene 9a

This compound was synthesized from resin 7a (997 mg, 1.21 mmol) and 4-phenyl phenol as described in Example 4. Solid phase extraction (50:10 heptane/ethyl acetate) gave 285 mg (83%) of the desired product as a solid (84% purity by GC, Rt=12.7 min). An analytical sample was obtained by flash chromatography (40:10 heptane/ethyl acetate). Recrystallisation from heptane gave white crystalls: mp 62-63° C. TLC: R_{f}=0.17. ¹H NMR: δ1.22 (t, 3H, J=7.0), 3.54 (q, 2H, J=7.0), 3.62 (t, 2H, J=4.8), 3.73 (t, 2H, J=4.8), 3.88 (t, 2H, J=4.9), 4.17 (t, 2H, J=4.9), 6.98 (d, 2H, J=8.6), 7.28 (t, 1H, J=7.3), 7.40 (t, 2H, J=7.6), 7.51 (d, 2H, J=8.6), 7.54 (d, 2H, J=7.6). ¹³C NMR: δ15.6, 67.1, 67.9, 70.2, 70.3, 71.4, 115.3, 127.1, 127.2, 128.5, 129.1, 134.3, 141.2, 158.8. HRMS: calcd. for C₁₈H₂₂O₃ 286.1569, found 286.157. Anal. Calcd. for C₁₈H₂₂O₃: C, 75.50; H, 7.74, found C, 75.27; H, 8.00.

### Example 9

Hexyloxy-4-phenylbenzene 10a

This compound was synthesized from resin 8a (1.02 g, 1.29 mmol) and 4-phenylphenol as described in Example 4. Solid phase extraction (150:10 heptane/ethyl acetate) gave 262 mg (80%) of the desired product as a solid (87% purity by GC, Rt=11.7 min). An analytical sample was obtained by flash chromatography (300:10 heptane/ethyl acetate). Recrystallisation from heptane gave white crystalls: mp 61-62° C. TLC: R_{f}=0.42. ¹H NMR: δ0.91 (t, 3H, J=6.2), 1.34 (m, 4H), 1.46 (m, 2H), 1.78 (p, 2H, J=7.1), 3.97 (t, 2H, J=6.6), 6.94 (d, 2H, J=8.5), 7.27 (t, 1H, J=7.4), 7.38 (t, 2H, J=7.6), 7.49 (d, 2H, J=8.5), 7.53 (d, 2H, J=7.8). ¹³C NMR: δ14.5, 23.1, 26.2, 29.7, 32.1, 68.5, 115.3, 127.0, 127.1, 128.5, 129.1, 134.0, 141.4, 159.2. HRMS: calcd. for C₁₈H₂₂O 254.1670, found 254.166. Anal. Calcd. for C₁₈H₂₂O: C, 84.99; H, 8.72, found C, 84.73; H, 8.97.

What is claimed is:

1. A solid support for solid phase synthesis of a combinatorial library of organic compounds, wherein the solid support comprises polystyrene bound selenium.

2. A solid support having the formula PS—Se—B(OR)₃⁻M⁺, wherein PS is polystyrene; R is C₁-C₆ alkyl; M is Li, Na, K, Zn or Cs.

3. A solid support according to claim 2, wherein R is ethyl.

4. A solid support according to claim 2, wherein M is Na.

5. A process for preparing a solid support according to claim 1, wherein the process comprises the steps of:
a) lithium-bromine exchange of bromopolystyrene with BuLi;
b) suspension in a non-protic solvent and treatment with selenium, wherein the polar non-protic solvent is selected from the group consisting of dimethoxyethan, diethylether, THF, toluene or dioxane, and
c) treatment with Mⁿ⁺(BH₄)ₙ⁻ in ROH.

6. The process of claim 5, wherein said polar non-protic solvent is THF.

7. A method for synthesizing organic compounds on a solid support according to claim 1, wherein the solid support comprises polystyrene bound selenium and the method comprises the steps of:
a) attachment by direct loading to the solid support of a compound of formula R¹R²R³CX, wherein X is a halogenide or a substituted alkyl or aryl sulfonate; R¹, R² and R³ are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl, provided that at least one of R¹, R² and R³ is not hydrogen;
b) additional modification of the R¹, R² or R³-groups by a synthesis sequence comprising one or more reactions being compatible with aryl alkyl selenides;
c) cleavage with formation of aliphatic C—H bond on final compound of formula R^{1′} R^{2′} R^{3′} CH wherein R^{1′}, R^{2′} and R^{3′} are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl; and
d) optionally purification by solid phase extraction.

8. The method of claim 7, wherein said organic compounds are a combinatorial library of compounds.

9. The method of claim 7, wherein the cleavage step of step (c) is a radical homolysis with trialkyl stannanes and a radical initiator.

10. The method of claim 9, wherein said radicial initiator is AIBN.

11. A method for synthesizing organic compounds on a solid support according to claim 1, wherein the solid support is polystyrene bound selenium and the method comprises the steps of:
a) attachment by direct loading to the solid support of a compound of formula XCR¹R²—CHR³R⁴, wherein X is a halogenide or a substituted alkyl or aryl sulfonate; R¹, R², R³ and R⁴ are the same or different and are hydrogen, optionally substituted alkyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl, provided that at least one of R¹, R², R³ and R⁴ is not hydrogen;
b) additional modification of the R¹, R², R³ and R⁴-groups by a synthesis sequence comprising one or more reactions being compatible with aryl alkyl selenides;
c) cleavage under oxidative condition under β-elimination process on final compounds of the general structure CR^{1′}R^{2′}═CR^{3′}R^{4′}, wherein R^{1′}, R^{2′}, R^{3′} and R^{4′} are the same or different and are hydrogen, optionally substituted alkyl, optionally substituted cycloalkyl, optionally substituted alkenyl, optionally substituted alkynyl, optionally substituted cycloalkyl, optionally substituted alkenyl, optionally substituted alkynyl, optionally substituted aryl, optionally substituted heteroaryl, optionally substituted arylalkyl, optionally substituted heteroarylalkyl, optionally substituted heterocyclic, optionally substituted heteroalkyl, optionally substituted heterocyclicalkyl or optionally substituted alkylheteroalkyl; and
d) optionally purification by solid phase extraction.

12. The method of claim 11, wherein said organic compounds are a combinatorial library of compounds.

13. The method of claim 11, wherein the cleavage of step c) is a oxidation with sodium periodide, H₂O₂ or m-CPBA.

14. A method for synthesizing organic compounds on a solid support wherein the solid support comprises polystyrene bound selenium.
